(19) United States
(12) Patent Application Publication
(10) Pub. No.: US 2026/0351352 A1
(43) Pub. Date: Nov. 5, 2026

(54) PREDICTION DEVICE, DATA PROCESSING DEVICE, PREDICTION METHOD, DATA PROCESSING METHOD, COMPUTER PROGRAM, AND RECORDING MEDIUM

(71) Applicant: Panasonic Intellectual Property Management Co., Ltd., Osaka (JP)

(72) Inventors: Daijiro NOZAKI, Osaka (JP); Nobuyuki MATSUZAWA, Tokyo (JP); Hiroyuki MAESHIMA, Hyogo (JP)

(21) Appl. No.: 19/099,346

(22) PCT Filed: Jul. 25, 2023
(86) PCT No.: PCT/JP2023/027290

§ 371 Date: Jan. 28, 2025

(30) Foreign Application Priority Data
Aug. 10, 2022 (JP) 2022-128523

(51) Int. Cl. G16C 20/30

(52) U.S. Cl. CPC G16C 20/30

(57) ABSTRACT

Z predictive models are associated one-to-one with Z explanatory variable sets differing from each other. The Z explanatory variable sets are each a combination of p explanatory variables differing from each other from among N explanatory variables. The N explanatory variables represent a molecular structure. The Z predictive models each calculate a value of a response variable representing the property imparted by the subject molecular compound, by substituting a value corresponding to the molecular structure of the subject molecular compound into the explanatory variable set associated with the predictive model. A model selection portion selects, according to the value of the explanatory variable set corresponding to the molecular structure of the subject molecular compound, A predictive models from among the Z predictive models. A prediction performing portion predicts, with use of the A predictive models, the property imparted by the subject molecular compound.

### TECHNICAL FIELD

The present disclosure relates to a prediction device, a data processing device, a prediction method, a data processing method, a computer program, and a recording medium.

### BACKGROUND ART

In recent years, machine learning has been utilized in various fields (e.g., Patent Literatures 1 and 2). One example of a technology utilizing machine learning is materials informatics (MI).

### CITATION LIST

### Patent Literature

Patent Literature 1: JP 2018-77547 A
Patent Literature 2: JP 2006-048429 A

### Non Patent Literature

Non Patent Literature 1: R. Ramprasad et al. npj Computational Materials 3, 54 (2017).
Non Patent Literature 2: Y. Mo, S. P. Ong and G. Ceder, Chem. Mat. 24, 15, (2012).

### SUMMARY OF INVENTION

### Technical Problem

The present disclosure provides a technique suitable for performing prediction of a property imparted by a molecular compound according to the molecular structure of the molecular compound.

### Solution to Problem

The present disclosure provides a prediction device configured to predict, from a molecular structure of a subject molecular compound, at least one property imparted by the subject molecular compound, the prediction device including:
Z trained predictive models, where Z is a natural number greater than or equal to 2, the Z predictive models being associated one-to-one with Z explanatory variable sets differing from each other, the Z explanatory variable sets each being a combination of p explanatory variables differing from each other from among N explanatory variables, where N is a natural number greater than or equal to 2, the N explanatory variables representing a molecular structure, p is a natural number greater than or equal to 1 and less than N, the Z predictive models each being configured to calculate a value of a response variable representing the property imparted by the subject molecular compound, by substituting a value corresponding to the molecular structure of the subject molecular compound into the explanatory variable set associated with the predictive model;
a model selection portion configured to select, according to the value of the explanatory variable set corresponding to the molecular structure of the subject molecular compound, A predictive models from among the Z predictive models, where A is a natural number greater than or equal to 1 and less than Z; and
a prediction performing portion configured to predict, with use of the A predictive models, the property imparted by the subject molecular compound.

### Advantageous Effects of Invention

The technique according to the present disclosure is suitable for performing prediction of a property imparted by a molecular compound according to the molecular structure of the molecular compound.

### BRIEF DESCRIPTION OF DRAWINGS

FIG. 1 is a block diagram of a data processing device of an embodiment.

FIG. 2 is a flowchart for illustrating the processing performed by a data processing device of the embodiment.

FIG. 3 shows an example of molecular compound data according to the embodiment.

FIG. 4 is a scatter plot showing the relationship between R_{training}² of a predictive model according to an example and R_{validation}² of the same predictive model.

FIG. 5A is a scatter plot showing the relationship between the actual values of the flash point and the predicted values of the flash point obtained in the case of using a single model having the highest R_{validation}².

FIG. 5B is a scatter plot showing the relationship between the actual values of the flash point and the predicted values of the flash point obtained in the case of constructing an ensemble model for each organic molecular compound by selecting predictive models according to the molecular structure of the organic molecular compound.

FIG. 6 is a scatter plot showing the distribution of the predicted values of the flash points of the molecular compounds contained in the QM9 library, predicted by a prediction device according to the example.

FIG. 7 shows the top 5 molecular compounds having the highest flash points together with the predicted values of their flash points, when molecular compounds that can be proposed in the example are arranged in descending order of flash points.

### DESCRIPTION OF EMBODIMENTS

### (Findings by the Present Inventors)

Various efforts have been made in material development.

In recent years, artificial intelligence (AI) technologies based on machine learning and the like have advanced. Alongside this, research on material creation using materials informatics (MI) has become increasingly active. MI is a technology that integrates data science and materials science. In data science, data is collected. Such collected known data is provided to AI. This enables prediction for unknown data, enabling new knowledge systems to be constructed. In MI, such data science methods are applied to materials science to develop new materials based on accumulated past data. One specific example of MI is described in Non Patent Literature 1.

Conventional material development without relying on MI heavily relies on tacit knowledge, such as intuition and experience, of researchers. In conventional material development without relying on MI, data acquisition has sometimes involved repeating experiments hundreds or even thousands of times to achieve desired properties. This requires significant time, effort, and cost. In contrast, in material development using MI, data from databases is provided to AI, thereby providing highly accurate experimental candidates. Experiments related to the provided experimental candidates are conducted. By repeating the cycle of providing experimental candidates and conducting experiments related to the candidates, the period required for material development can be reduced. In a numerical example, the period may be reduced from ten years to approximately one or two years. Reducing the period leads to a reduction in effort and cost required for development. In practice, one company spent five years developing a certain all-solid-state battery material through material development without relying on MI. In contrast, another later company has completed the same all-solid-state battery material in one year through material development using MI. This example remains a prominent case of development period reduction achieved through MI. For further details on such advantages of MI, refer to Non Patent Literature 2.

Here, it is considered that a prediction device including X trained predictive models is used to predict, from the molecular structure of a molecular compound, a property imparted by the molecular compound, where X is a natural number greater than or equal to 2.

In a reference prediction example, from among the X predictive models, r predictive models having excellent values of a metric related to prediction accuracy are selected. The prediction of the property imparted by the molecular compound from its molecular structure is performed for each of a plurality of molecular compounds with use of the above r predictive models. With this, the property can be predicted with excellent accuracy in some cases. However, the prediction accuracy of predictions performed in this manner varies between molecular compounds. Depending on the molecular structure, there is a risk that sufficient prediction accuracy cannot be achieved.

Therefore, the present inventors have examined a technique suitable for performing prediction of a property imparted by a molecular compound according to the molecular structure of the molecular compound.

In the present specification, the expression “subject molecular compound” may be used. The expression “subject” is not intended to restrictively interpret the molecular compound.

In the present specification, the expression “molecular structure” may be used. “Molecular structure” is a concept that can encompass the number, type, arrangement, and bonding mode of constituent atoms within the molecule. Furthermore, in the present specification, the expression “molecular structure of a molecular compound” may be used. In this expression, the molecular structure may refer to the overall structure of the molecular compound or may refer to a partial structure of the molecular compound. The partial structure is, for example, a functional group. The same applies to expressions such as “molecular structure of a subject molecular compound”.

In the present specification, the expression “property imparted by a molecular compound” may be used. This expression may refer to a property of the molecular compound itself or may refer to a property of a device or the like containing the molecular compound and other elements. The same applies to expressions such as “at least one property imparted by a subject molecular compound”.

In the present specification, the expression “explanatory variable set” may be used. In this expression, the number of explanatory variables included in an explanatory variable set may be one or may be two or more.

In the present specification, the symbol “*” may be used to represent a vector.

For example, “x*” represents a vector.

### EMBODIMENTS

The present disclosure is described below through embodiments. The following embodiments do not limit the disclosure according to the claims. Furthermore, all the combinations of the features described in the embodiments are not necessarily essential to the solution of the present disclosure.

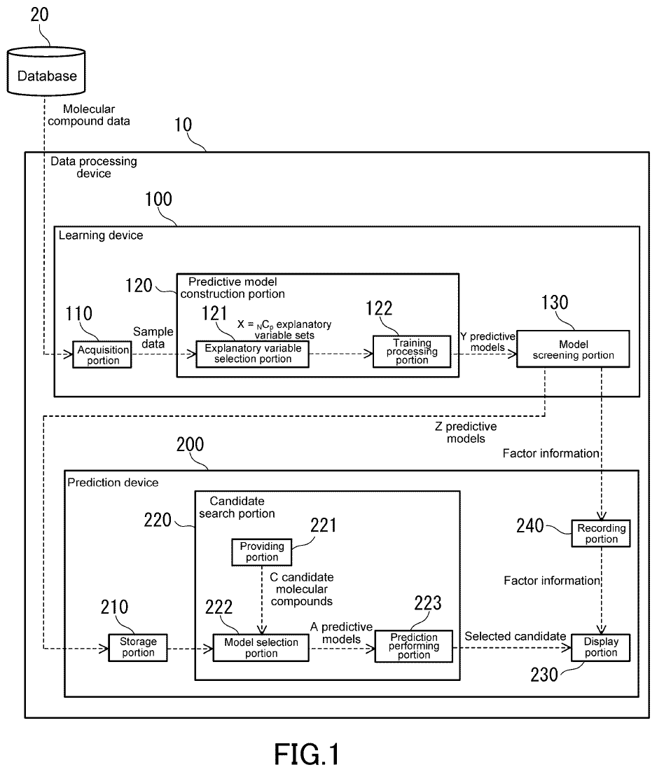

FIG. 1 is a block diagram of a data processing device 10 of an embodiment. The data processing device 10 includes a learning device 100 and a prediction device 200. In the learning device 100, a plurality of explanatory variable sets are selected with use of a plurality of explanatory variables representing a molecular structure. Each explanatory variable set includes at least one explanatory variable. Next, a plurality of predictive models are constructed based on a plurality of sample data pieces related to a molecular compound. The predictive model is associated with the explanatory variable set. The predictive model is configured to determine the value of a response variable representing at least one property imparted by the subject molecular compound, by substituting a value corresponding to the molecular structure of the subject molecular compound into the explanatory variable set associated with the predictive model.

In the prediction device 200, data is generated that represents the molecular structures of a plurality of molecular compounds that are candidates (hereinafter, candidate molecular compounds). Next, one or more predictive models are selected from among the plurality of predictive models. Next, with use of the selected predictive models, at least one property imparted by the candidate molecular compound is predicted. When the property predicted to be imparted by a candidate molecular compound is within a target range, the candidate molecular compound is proposed. The proposal is directed to a user of the prediction device 200 or the data processing device 10. Specifically, the proposal is performed by displaying the information.

As understood from the above description, the data processing device 10 predicts that the property represented by the response variable is imparted by the factor represented by each explanatory variable. The value of the response variable may be an experimentally measured value or a theoretically calculated value.

The learning device 100 includes an acquisition portion 110, a predictive model construction portion 120, and a model screening portion 130. The prediction device 200 includes a storage portion 210, a candidate search portion 220, a display portion 230, and a recording portion 240. The selection of explanatory variable sets and the construction of predictive models are performed by the predictive model construction portion 120. The generation of data representing the molecular structures of candidate molecular compounds, the selection of predictive models, and the prediction of properties of candidate molecular compounds are performed by the candidate search portion 220. The display of candidate molecular compounds is performed by the display portion 230.

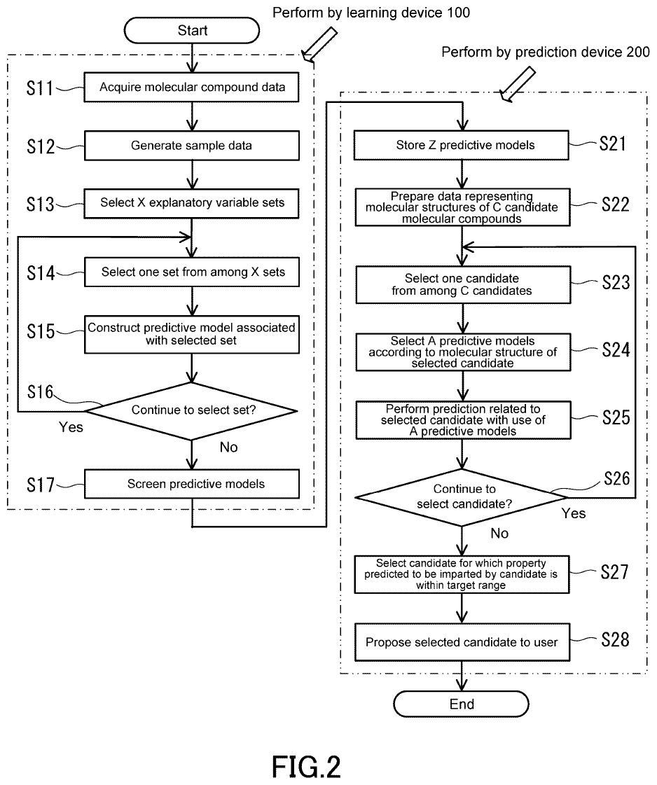

The predictive model construction portion 120 includes an explanatory variable selection portion 121 and a training processing portion 122. The candidate search portion 220 includes a providing portion 221, a model selection portion 222, and a prediction performing portion 223. The following describes in detail, with reference to FIG. 2, the acquisition portion 110, the explanatory variable selection portion 121, the training processing portion 122, the model screening portion 130, the storage portion 210, the providing portion 221, the model selection portion 222, the prediction performing portion 223, the display portion 230, and the recording portion 240. FIG. 2 is a flowchart for illustrating the processing performed by the data processing device 10 of the embodiment. Steps S11 to S17 are performed by the learning device 100. Steps S21 to S28 are performed by the prediction device 200.

In Step S11, the acquisition portion 110 acquires a plurality of molecular compound data pieces from the database 20. In each molecular compound data piece, the molecular structure of a molecular compound and a property imparted by the molecular compound are associated with each other. In the present embodiment, in each molecular compound data piece, the property imparted by the molecular compound can be a property obtained by measurement or a property obtained theoretically.

Next, in Step S12, the acquisition portion 110 generates a plurality of sample data pieces. Specifically, the acquisition portion 110 converts the molecular structure of the molecular compound in each molecular compound data piece, using molecular descriptors. Thus, values of a plurality of explanatory variables representing the molecular structure of the molecular compound are obtained. Furthermore, the value of the property imparted by the molecular compound in each molecular compound data piece is used as the value of the response variable. Thus, the plurality of sample data pieces are obtained. In each sample data piece, the values of the plurality of explanatory variables representing the molecular structure of the molecular compound and the value of the response variable representing the property imparted by the molecular compound are associated with each other. In the present embodiment, in each sample data piece, the value of the response variable representing the property imparted by the molecular compound can be a property value obtained by measurement or a property value obtained theoretically. The plurality of sample data pieces are provided to the predictive model construction portion 120.

The molecular descriptors can be calculated using a tool such as RDKit, mordred, Dragon7, cinfony, PaDEL-descriptor, or Chemopy. For the input values of the molecular descriptors, for example, the simplified molecular input line entry system (SMILES) notation is used, which is a notation method that converts the chemical structure of molecules into alphanumeric ASCII character strings without structural ambiguity. RDKit, mordred, Dragon7, cinfony, PaDEL-descriptor, and Chemopy can calculate 208, 1,825, 5,270, 307, 797, and 252 types of molecular descriptors, respectively. The molecular descriptors may be custom-made molecular descriptors.

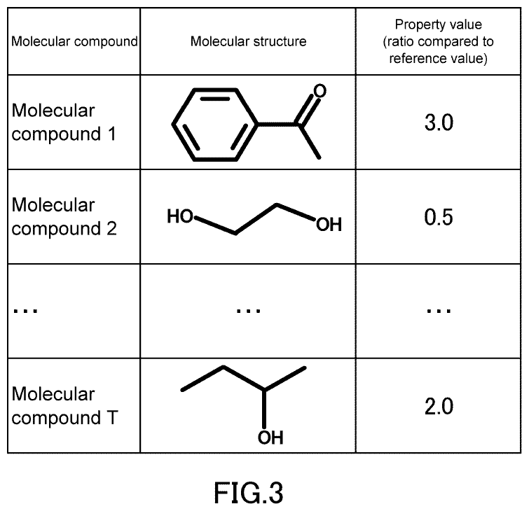

FIG. 3 shows an example of the molecular compound data according to the embodiment. In FIG. 3, T molecular compound data pieces are listed. Each row in FIG. 3 corresponds to one molecular compound data piece. The T molecular compound data pieces correspond one-to-one to T molecular compounds differing from each other. The T molecular compounds are specifically Molecular compound 1, Molecular compound 2, . . . , and Molecular compound T. FIG. 3 shows that
Molecular compound 1 yields a property value of 3.0 based on its molecular structure,
Molecular compound 2 yields a property value of 0.5 based on its molecular structure,
Molecular compound T yields a property value of 2.0 based on its molecular structure.
   In the example in FIG. 3, Molecular compound 1, Molecular compound 2, . . . , and Molecular compound T are specifically organic molecular compounds. The property value is normalized by dividing the actual value by a reference value.

The property may be a physical property of the subject molecular compound itself, a physical property of a material containing the molecular compound, or a device property of a device containing the molecular compound. The property is, for example,
flash point,
viscosity,
solubility,
resistance,
boiling point,
melting point
dielectric constant,
current,
voltage,
current-voltage characteristics,
operating frequency of an electronic device,
operating characteristics of an electronic device,
degree of degradation of an electronic device, or
shape of an electronic device.

As described above, the property may be an experimentally measured property, a theoretically calculated property, or a property obtained by other methods. When a plurality of properties are predicted, a predictive model is constructed for each property, and a plurality of elements (e.g., flash point and viscosity) from among the above list can be used as the plurality of properties.

Next, in Step S13, the explanatory variable selection portion 121 selects X explanatory variable sets differing from each other. Specifically, in the present embodiment, the molecular structure of a molecular compound is represented by N explanatory variables. The explanatory variable selection portion 121 selects X explanatory variable sets so that the X explanatory variable sets are each a combination of p explanatory variables from among the N explanatory variables, where N is a natural number greater than or equal to 2, X is a natural number greater than or equal to 2, and p is a natural number greater than or equal to 1 and less than N. The p explanatory variables are specifically p explanatory variables differing from each other. “X explanatory variable sets differing from each other” means that at least one explanatory variable differs between the p explanatory variables in one explanatory variable set and the p explanatory variables in another explanatory variable set. For example, assume a case where p is 4, the p explanatory variables in one explanatory variable set are x₁, x₂, x₃, and x₄, and the p explanatory variables in another explanatory variable sets are x₁, x₂, x₃, and x₅. In this case, the explanatory variables in these sets can be said to differ from each other.

In one specific example, X=_{N}Cₚ. In this case, the X explanatory variable sets can be selected to cover all combinations of selecting the p explanatory variables from among the N explanatory variables. However, to satisfy X<_{N}Cₚ, the combination of the p explanatory variables from among the N explanatory variables may be selected randomly, for example. N is, for example, greater than or equal to 3 and less than or equal to 1,000,000. In one specific example, N is greater than or equal to 3 and less than or equal to 10,000, and in another specific example, N is greater than or equal to 10,000 and less than or equal to 1,000,000. The value of p is, for example, greater than or equal to 2 and less than or equal to 10,000,000. In one specific example, p is greater than or equal to 2 and less than or equal to 20, and in another specific example, p is greater than or equal to 3 and less than or equal to 10,000,000. X is, for example, greater than or equal to 3 and less than or equal to 10,000,000,000,000,000. In one specific example, X is greater than or equal to 3 and less than or equal to 10,000,000, and in another specific example, X is greater than or equal to 50,000 and less than or equal to 10,000,000,000,000,000.

Next, in Step S14, the explanatory variable selection portion 121 selects one explanatory variable set from among the X explanatory variable sets.

Next, in Step S15, the training processing portion 122 constructs a predictive model associated with the one explanatory variable set selected in Step S14. Specifically, the plurality of sample data pieces above are used as a plurality of training data pieces. In each training data piece, the values of a plurality of explanatory variables representing the molecular structure of a molecular compound and the value of a response variable representing the property imparted by the molecular compound are associated with each other. In the present embodiment, in each training data piece, the value of the response variable representing the property imparted by the molecular compound can be a property value obtained by measurement or a property value obtained theoretically. The training processing portion 122 constructs a predictive model associated with the explanatory variable set selected in Step S14, by training with use of the plurality of training data pieces. In the present embodiment, the training performed by the training processing portion 122 is supervised learning.

The training for constructing the predictive model is based on, for example, at least one selected from the group consisting of:
a linear function;
a function regularized using ridge regression;
a function regularized using Lasso regression;
a support vector machine;
a neural network;
a maximum likelihood estimation method; and
a Bayesian method.
   Typically, support vector machines and neural networks are nonlinear functions. The neural networks may be based on a self-organizing map. The training may be deep learning with use of a neural network. The training may be by multivariate analysis, such as discriminant analysis or multiple regression analysis.

To describe using mathematical expressions, in the present embodiment, the predictive model is represented by a function f(x*). The value x*={x₁, x₂, . . . , xₚ} is the input vector of the function f(x*). The function f(x*) may be an analytic function that takes each element of the vector x* as an input value. The analytic function is, for example, a linear function or a regularized function. The function f(x*) may be a nonlinear function that takes each element of the vector x* as an input value. The nonlinear function is, for example, a support vector machine or a neural network. Each element of the vector x* can be based on a molecular descriptor.

In a first modeling example, p=3. The function f(x*) is a linear function. The explanatory variable set includes explanatory variables corresponding to the 11th, 15th, and 34th RDKit molecular descriptors. The function f(x*) is a linear function in a three-dimensional space. Specifically, f(x*) includes each element of the vector x* represented in a three-dimensional space having respective axes defined by the above RDKit molecular descriptors. More specifically,

f(x*)=a₁₁x₁₁+a₁₅x₁₅+a₃₄x₃₄+const₁,

where a₁₁, a₁₅, and a₃₄ are coefficients, const₁ is a constant term, and x₁₁, x₁₅, and x₃₄ are respectively explanatory variables corresponding to the 11th, 15th, and 34th RDKit molecular descriptors of the molecular compound. The values of the coordinates (x₁₁,x₁₅,x₃₄) represented by the training data are substituted into the function f(x*) to obtain the value of f(x*) that is the output value. By performing this for a plurality of training data pieces, a plurality of output values are obtained. These output values are the predicted values representing the property value imparted by the molecular compound, predicted by the function f(x*). On the other hand, the value of the response variable for each of the plurality of training data pieces is the actual value representing the property value. The coefficients all, a₁₅, and a₃₄ are optimized so that the sum of the squares of the absolute errors between the predicted values and the actual values for the plurality of training data pieces is minimized. This determines the function f(x*).

In a second modeling example, the function f(x*) includes intersection terms of different molecular descriptors. Specifically,

f(x*)=a₁₁x₁₁+a₁₅x₁₅+a₃₄x₃₄+b_{11,15}×x₁₁×x₁₅+b_{11,34}x₁₁x₃₄+b_{15,34×15×34}+const₂,

where b_{11,15}, b_{11,34}, and b_{15,34} are coefficients, and const₂ is a constant term. The values of the coordinates (x₁₁,x₁₅,x₃₄) represented by the training data are substituted into the function f(x*) to obtain the value of f(x*) that is the output value. By performing this for a plurality of training data pieces, a plurality of output values are obtained. These output values are the predicted values representing the property value imparted by the molecular compound, predicted by the function f(x*). On the other hand, the value of the response variable for each of the plurality of training data pieces is the actual value representing the property value. The coefficients all, a₁₅, a₃₄, b_{11,15}, b_{11,34}, and b_{15,34} are optimized so that the sum of the squares of the absolute errors between the predicted values and the actual values for the plurality of training data pieces is minimized. This determines the function f(x*).

In a third modeling example, the function f(x*) includes square terms of molecular descriptors. Specifically,

f(x*)=a₁₁x₁₁+a₁₅x₁₅+a₃₄x₃₄+b_{11,15}x₁₁x₁₅+b_{11,34×11×34}+b_{15,34×15×34}+c₁₁x₁₁²+c₁₅x₁₅²+c₃₄x₃₄²+const₃,

where c₁₁, c₁₅, and c₃₄ are coefficients, and const₃ is a constant term. The values of the coordinates (x₁₁,x₁₅,x₃₄) represented by the training data are substituted into the function f(x*) to obtain the value of f(x*) that is the output value. By performing this for a plurality of training data pieces, a plurality of output values are obtained. These output values are the predicted values representing the property value imparted by the molecular compound, predicted by the function f(x*). On the other hand, the value of the response variable for each of the plurality of training data pieces is the actual value representing the property value. The coefficients a₁₁, a₁₅, a₃₄, b_{11,15}, b_{11,34}, b_{15,34}, c₁₁, c₁₅, and c₃₄ are optimized so that the sum of the squares of the absolute errors between the predicted values and the actual values for the plurality of training data pieces is minimized. This determines the function f(x*).

In a fourth modeling example, the function f(x*) is a nonlinear function of x* based on a plurality of molecular descriptors. The values of each element of the vector x* represented by the training data are substituted into the function f(x*) to obtain the value of f(x*) that is the output value. By performing this for a plurality of training data pieces, a plurality of output values are obtained. These output values are the predicted values representing the property value imparted by the molecular compound, predicted by the function f(x*). On the other hand, the value of the response variable for each of the plurality of training data pieces is the actual value representing the property value. The function f(x*) is optimized so that the sum of the squares of the absolute errors between the predicted values and the actual values for the plurality of training data pieces is minimized. This determines the function f(x*). Specifically, in the fourth modeling example, the function f(x*) can be a neural network or a support vector machine. When the function f(x*) is a neural network, each weight coefficient and bias term are optimized. When the function f(x*) is a support vector machine, each weight coefficient and constant term are optimized.

In Step S15 of the present embodiment, the predictive model is constructed, and furthermore the accuracy of the predictive model is evaluated. The number of sample data pieces is hereinafter denoted as J, where J is a natural number greater than or equal to 3. As understood from the above description, the training processing portion 122 constructs a predictive model associated with a selected explanatory variable set, by training with use of a plurality of training data pieces. In the present embodiment, in constructing a predictive model, all the J sample data pieces are used for training as the plurality of training data pieces above.

In contrast, in evaluating the accuracy of the predictive model, from among the J sample data pieces, V pieces are used as validation data and the remaining L pieces are used as training data, where V is a natural number greater than or equal to 1, and L is a natural number greater than or equal to 2. Specifically, the training processing portion 122 divides the J sample data pieces into M sets, where M is a natural number greater than or equal to 2. One set of the M sets includes V validation data pieces. M−1 sets of the M sets include L training data pieces as a whole. The M−1 sets of the M sets each include a plurality of training data pieces. The number of training data pieces included in each of the M−1 sets may or may not be exactly the same. The number that represents the number of training data pieces in each of the M−1 sets and may or may not be exactly the same across the M−1 sets is hereafter denoted as [d], where [d] is a natural number greater than or equal to 2. The sample data is divided in this manner and cross-validation is performed. In the process of cross-validation, the sets used as validation data is sequentially changed, and consequently, M evaluation models are constructed for each predictive model. In other words, M evaluation models are constructed for each set of molecular descriptors.

### <Acquisition of Validation-Data-Based Evaluation of Predictive Model>

Then, the training processing portion 122 substitutes, for each of the M evaluation models, a value corresponding to the validation data into the p explanatory variables. The resulting value of the response variable is the predicted value representing the property value imparted by the molecular compound relating to the validation data. On the other hand, the value of the response variable for the validation data is the actual value representing the property value. Thus, M×V predicted value-actual value pairs are obtained. Based on these predicted value-actual value pairs, the validation-data-based evaluation of the predictive model is acquired.

### <Acquisition of Training-Data-Based Evaluation of Predictive Model>

Moreover, the training processing portion 122 substitutes, for each of the M evaluation models, a value corresponding to the training data into the p explanatory variables. The resulting value of the response variable is the predicted value representing the property value imparted by the molecular compound relating to the training data. On the other hand, the value of the response variable for the training data is the actual value representing the property value. Thus, M×[d] predicted value-actual value pairs are obtained. Based on these predicted value-actual value pairs, the training-data-based evaluation of the predictive model is acquired.

Leave-one-out cross-validation may be performed with V=1.

The evaluation metric for the training-data-based-evaluation of the predictive model is not limited to any particular metric. Examples of the evaluation metric include the following evaluation metrics calculated based on the M×[d] predicted value-actual value pairs:
the sum of the absolute errors between the actual values and the predicted values (MAE);
the standard deviation of the errors between the actual values and the predicted values; and
a training-data-based coefficient of determination R_{training}² obtained from the distribution of the actual values and the predicted values.
   Specifically, these evaluation metrics are evaluation metrics for prediction accuracy.

The evaluation metric for the validation-data-based-evaluation of the predictive model is not limited to any particular metric. Examples of the evaluation metric include the following evaluation metrics calculated based on the M×V predicted value-actual value pairs:
the sum of the absolute errors between the actual values and the predicted values (MAE);
the standard deviation of the errors between the actual values and the predicted values; and
a validation-data-based coefficient of determination R_{validation}² obtained from the distribution of the actual values and the predicted values.
   Specifically, these evaluation metrics are evaluation metrics for prediction accuracy.

As an evaluation metric for the comprehensive prediction accuracy of the predictive model, for example, at least one selected from the group consisting of the above coefficient of determination R_{training}² and the above coefficient of determination R_{validation}² can be adopted. In the present embodiment, as an evaluation metric for the comprehensive prediction accuracy of the predictive model, a set of coefficients of determination R² (R_{training}², R_{validation}²) that is a combination of the above coefficient of determination R_{training}² and the above coefficient of determination R_{validation}² is adopted.

As understood from the above description, in Step S15 of the present embodiment, the values of the sample data can be applied to a multidimensional space having axes defined by a plurality of molecular descriptors. The coefficients associated with the selected plurality of explanatory variables in the predictive model based on a linear function or the like can be adjusted so that the sum of the squares of the errors with respect to the actual values is minimized. Through such training, a predictive model capable of property prediction can be constructed. Furthermore, the prediction accuracy of the predictive model can be evaluated with use of the set of coefficients of determination R² (R_{training}², R_{validation}²).

Next, in Step S16, the predictive model construction portion 120 judges whether to continue to select an explanatory variable set. If it is judged to continue, the flow proceeds to Step S14. Then, in Step S14, an explanatory variable set is selected that differs from the explanatory variable sets previously selected. If it is judged not to continue, the flow proceeds to Step S17.

In Step S16 of the present embodiment, if neither a first condition nor a second condition is satisfied, it is judged to continue. If at least one selected from the group consisting of the first condition and the second condition is satisfied, it is judged not to continue. The first condition is a condition that the number of executions of Step S15 has reached a specified number of times. The specified number of times may be X times or less than X times. The second condition is a condition that the number of predictive models constructed in Step S15 and evaluated to have an evaluation greater than or equal to a threshold value has reached a threshold count. In the present embodiment, the threshold count is 2 or more.

At the stage when Step S17 is reached, Y predictive models have been constructed, where Y is a natural number greater than or equal to 2 and less than or equal to X.

In Step S17, the model screening portion 130 screens the predictive models. Specifically, the model screening portion 130 selects a predictive model that satisfies a screening condition from among the Y predictive models. In the present embodiment, the screening condition includes an overfitting condition that the predictive model is not overfitted and an underfitting condition that the predictive model is not underfitted. Specifically, if R_{training}² is greater than R_{validation}² by at least an overfitting threshold value, the predictive model is judged to be overfitted. If R_{validation}² is greater than R_{training}² by at least an underfitting threshold value, the predictive model is judged to be underfitted.

The overfitting threshold value is, for example, greater than or equal to 0.05 and less than or equal to 0.40, with one numerical example being 0.15. The underfitting threshold value is, for example, greater than or equal to 0.05 and less than or equal to 0.40, with one numerical example being 0.15. The overfitting threshold value may be greater than the underfitting threshold value, equal to the underfitting threshold value, or less than the underfitting threshold value. To apply stricter judgment on overfitting, a smaller value can be set as the overfitting threshold value. To apply more lenient judgment on overfitting, a larger value can be set as the overfitting threshold value. To apply stricter judgment on underfitting, a smaller value can be set as the underfitting threshold value. To apply more lenient judgment on underfitting, a larger value can be set as the underfitting threshold value.

The screening condition may include an additional condition, in addition to the overfitting condition and the underfitting condition. Specifically, in the present embodiment, the screening condition includes the overfitting condition, the underfitting condition, and a correlation condition. The correlation condition is a condition that R_{validation}² is greater than a correlation threshold value. The correlation threshold value is, for example, greater than or equal to 0.1 and less than or equal to 0.9, with one numerical example being 0.4.

Through the screening in Step S17, Z predictive models are selected, where Z is a natural number greater than or equal to 2 and less than or equal to Y.

Next, in Step S21, the storage portion 210 stores Z predictive models. The Z predictive models form a population of predictive models.

Next, in Step S22, the providing portion 221 prepares data representing the molecular structures of C candidate molecular compounds, where C is a natural number greater than or equal to 1. In the present embodiment, C is a natural number greater than or equal to 2. Typically, the candidate molecular compound is a molecular compound having an unknown property.

In the present embodiment, the providing portion 221 can include a molecular generator. The molecular generator generates data representing the molecular structure of the candidate molecular compound. Specifically, the molecular generator repeatedly performs a process of randomly arranging a predesignated number of atoms selected from among a plurality of predesignated specified atomic species, randomly generating chemical bonds between the plurality of atoms, and then collecting the resulting molecule as the candidate molecular compound if the molecule is chemically valid.

The process of collecting a chemically valid molecule as the candidate molecular compound is performed in accordance with a specified algorithm such that a specified rule defining a permissible molecular structure is satisfied. The specified rule includes, for example, a rule related to the number of bonds between atoms. For example, the number of bonds between hydrogen and other atoms is 1, the number of bonds between nitrogen and other atoms is 3, and the number of bonds between carbon and other atoms is 4.

The providing portion 221 may collect the candidate molecular compound from a known database. Examples of the database include Pubchem, CAS, and ChemSpider. Typically, the candidate molecular compound that may be collected from the database is a molecular compound that has not been used by the training processing portion 122 for constructing the predictive model.

Next, in Step S23, the providing portion 221 selects one candidate molecular compound from among the C candidate molecular compounds.

Next, in Step S24, the model selection portion 222 selects A predictive models from among the population of predictive models according to the molecular structure of the candidate molecular compound selected in Step S23, where A is a natural number greater than or equal to 1 and less than Z. In the present embodiment, A is a natural number greater than or equal to 2 and less than Z. In the present embodiment, A is a predetermined number.

In the present embodiment, the A predictive models are selected in accordance with a selection criterion. The selection criterion is to preferentially select, from among predictive models that satisfy an interpolation condition, predictive models having higher evaluation. By including the interpolation condition in the selection criterion, predictive models suited to the molecular structure of the molecular compound can be selected. This can contribute to excellent prediction of the property. Preferentially selecting predictive models having higher evaluation can also contribute to excellent prediction of the property. In the present embodiment, the interpolation condition is a condition that, in a p-dimensional explanatory variable space, a candidate coordinate is interpolated relative to a reference coordinate group. The candidate coordinate refers to a coordinate defined by the value of the explanatory variable set corresponding to the molecular structure of the candidate molecular compound. The reference coordinate group is obtained by plotting, for each of the plurality of training data pieces used for training the predictive model, a coordinate defined by the value of the explanatory variable set corresponding to the training data piece. Typically, the above evaluation is an evaluation of prediction accuracy. Specifically, in the present embodiment, the above evaluation is the coefficient of determination R_{validation}².

Instead of the interpolation condition, a different condition may be used that indicates the relationship between the candidate coordinate and the reference coordinate group. The different condition is, for example, a distance condition. Specifically, in a p-dimensional explanatory variable space, from among the reference coordinate group, a reference coordinate having the shortest distance to the candidate coordinate is defined as a specific coordinate. Furthermore, the distance between the candidate coordinate and the specific coordinate is defined as a specific distance. The distance condition is a condition that the specific distance is less than or equal to a specified value. The above evaluation does not need to be the coefficient of determination R_{validation}². The above evaluation may be the sum of the absolute errors between the actual values and the predicted values (MAE) for the validation data, or may be the standard deviation of the errors between the actual values and the predicted values for the validation data.

If the number of predictive models satisfying the selection criterion is less than A in Step S24, an appropriate operation can be performed. In a first example, in this case, the flow returns to Step S17. Then, at least one selected from the group consisting of processing of increasing the overfitting threshold value, processing of increasing the underfitting threshold value, and processing of decreasing the correlation threshold value is performed, and then Step S17 and the steps subsequent thereto are performed again. In a second example, in this case, the steps subsequent to Step S24 are performed with use of the fewer than A predictive models selected in accordance with the selection criterion in Step S24. If the second example is adopted, “A” in the description of the steps subsequent to Step S24 is replaced with the above number that is less than A.

Next, in Step S25, the prediction performing portion 223 performs prediction related to the candidate molecular compound selected in Step S23, with use of the A predictive models selected in Step S24. Specifically, the prediction performing portion 223 predicts the property imparted by the candidate molecular compound, with use of the A predictive models. This property may be a property of the candidate molecular compound itself. This property may be a property of a device containing the candidate molecular compound and other elements.

In the present embodiment, A is greater than or equal to 2. The prediction performing portion 223 predicts the property imparted by the candidate molecular compound, with use of an ensemble model including A predictive models. Specifically, the predicted value of the property is calculated by each of the A predictive models. The mean of the A predicted values obtained is regarded as the predicted value of the property predicted by the ensemble model. Thus, the prediction using the ensemble model is performed. The mean is, for example, an arithmetic mean, a harmonic mean, a geometric mean, or a trimmed mean.

Next, in Step S26, the prediction performing portion 223 judges whether the number of executions in Step S25 has reached C times. If it is judged that the number of executions has reached C times, the flow proceeds to Step S27. If it is judged that the number of executions has not reached C times, the flow proceeds to Step S23.

At the stage when Step S27 is reached, for each of B candidate molecular compounds, the property predicted to be imparted by the candidate molecular compound is within the target range, where B is an integer greater than or equal to 0 and less than or equal to C. In the present embodiment, B is a natural number greater than or equal to 1 and less than or equal to C.

In Step S27, the prediction performing portion 223 selects a candidate molecular compound for which the property predicted to be imparted by the candidate molecular compound is within the target range. In the present embodiment, the B candidate molecular compounds are selected.

Next, in Step S28, the prediction device 200 proposes the candidate molecular compounds selected in Step S27 to a user of the prediction device 200 or the data processing device 10. In this proposal, the prediction device 200 notifies the user of the candidate molecular compounds together with the respective properties predicted to be imparted by the candidate molecular compounds. In the present embodiment, the candidate molecular compounds selected in Step S27 and the respective properties predicted to be imparted by the candidate molecular compounds are displayed on the display portion 230.

Specifically, a target value is within the target range. Through Step S27, the B candidate molecular compounds are sorted in a table format or the like. The prediction performing portion 223 re-sorts the B candidate molecular compounds in descending order of proximity to the target value of the respective property values predicted to be imparted by the B candidate molecular compounds. In Step S28, the re-sorted B candidate molecular compounds are displayed on the display portion 230 together with the respective properties predicted to be imparted by the B candidate molecular compounds.

In one specific example, in Step S25, if the property predicted to be imparted by the candidate molecular compound is within the target range, the prediction performing portion 223 predicts additional information of the candidate molecular compound. The additional information includes, for example, the similarity between the candidate molecular compound and each of the molecular compounds corresponding to the training data pieces used for training the A predictive models. The additional information includes, for example, an evaluation metric for the A predictive models and an evaluation based on the evaluation metric. Other examples of information included in the additional information include the molecular weight of the candidate molecular compound, the ease of synthesis of the candidate molecular compound, and the registration number of the candidate molecular compound in the molecular database. The prediction of the additional information is performed typically by calculation. Then, in Step S28, the prediction performing portion 223 notifies the user of the additional information or displays the additional information on the display portion 230.

The display portion 230 is, for example, a display screen of a smartphone, a personal computer, or a television. The display screen is, for example, a liquid crystal display or an organic electroluminescence display.

As understood from the above description, in the data processing device 10 according to one specific example of the present embodiment, a plurality of predictive models can be selected by screening. For each candidate molecular compound, from among the selected plurality of predictive models, one or more predictive models can be selected that satisfy a condition dependent on the molecular structure of the candidate molecular compound. From the predicted values obtained by the selected predictive models, one or more candidate molecular compounds can be selected for which the respective properties predicted to be imparted by the one or more candidate molecular compounds are within the target range. According to this configuration, for each candidate molecular compound, a predictive model according to the molecular structure of the candidate molecular compound can be selected. Therefore, prediction according to the molecular structure, ideally prediction optimized individually for each molecular structure, is achieved. This enables the properties of candidate molecular compounds having various molecular structures to be predicted with high reliability across the candidate molecular compounds. The user can be notified of the selected candidate molecular compounds together with the properties imparted by the candidate molecular compounds, etc.

An advantage of constructing an ensemble model including a plurality of predictive models that are based on linear functions is described. Predictive models based on linear functions are categorized as weak predictors. Weak predictors can be constructed even with a limited number of training data pieces. Ensemble modeling can improve prediction accuracy. Therefore, according to an ensemble model including a plurality of predictive models based on linear functions, it is possible to predict a property imparted by a molecular compound with excellent accuracy, even when based on a limited number of training data pieces. In one numerical example, accurate prediction can be achieved with use of approximately 10 to approximately 1,000 training data pieces. In one example, the number of training data pieces is 10 or more and less than 1,000, and in one specific example, the number of training data pieces is 20 or more and less than 200.

Furthermore, it is also possible to utilize factor information that represents a factor predicted to contribute to the property. Specifically, in the present embodiment, as described above, the Z predictive models that satisfy the screening condition are selected from among the Y predictive models through screening performed by the model screening portion 130. The screening condition includes the overfitting condition that the predictive model is not overfitted, the underfitting condition that the predictive model is not underfitted, and the correlation condition that R_{validation}² is greater than the correlation threshold value. The overfitting condition, the underfitting condition, and the correlation condition are all metrics related to prediction accuracy. Accordingly, the Z predictive models can have higher prediction accuracy than the predictive models excluded through the screening. In the present embodiment, from among the N explanatory variables, an explanatory variable having the highest cumulative number of appearances in the Z explanatory variable sets corresponding to the Z predictive models is referred to as a specific explanatory variable. The factor information based on the specific explanatory variable has a high likelihood of contributing to the property. Accordingly, the information based on the specific explanatory variable is utilized as the factor information that represents the factor predicted to contribute to the property. Such utilization can contribute to the creation of a new molecular compound or the creation of a device containing such a molecular compound.

For example, the factor information is recorded in the recording portion 240 and/or displayed on the display portion 230. The recording portion 240 enables the factor information to be utilized later in various forms. The display portion 230 enables the user of the prediction device to review the factor information. For example, the factor information can be provided from the model screening portion 130 to the recording portion 240. The factor information can also be provided from the recording portion 240 to the display portion 230.

In the predictive model based on a linear function, it is easy to understand the correspondence between the explanatory variables related to the molecular structure and the response variable related to the property imparted by the molecular compound. This is advantageous from the viewpoint of increasing the likelihood that the factor information represents the factor that actually contributes to the property.

The technique according to the present disclosure is described below in more detail with reference to an example. In the example, a prediction device was configured in accordance with the above embodiment. Specifically, the configured prediction device can select, for each molecular compound, a plurality of predictive models based on linear functions according to the molecular structure of the molecular compound to construct an ensemble model including the selected predictive models. The prediction device was then used to search for a molecular compound having high flash point. When combustible liquids are used as materials for electronic devices, it is necessary to enhance safety from the viewpoints of the Fire Service Act and international distribution. Under the Fire Service Act, liquids having a flash point less than 250 °C are, in principle, regarded as hazardous materials. For this reason, it is desirable to use liquids having as high a flash point as possible. Many combustible liquids, depending on their flash points, can be classified as Class 4 hazardous materials (flammable liquids) under the Fire Service Act or as designated combustible materials (combustible liquids) under the same Act. Many organic solvents widely used in electronic devices and the like have flash points of 200 °C or less. For example, ethylene carbonate (EC), dimethyl carbonate (DMC), fluoroethylene carbonate (FEC), ethyl methyl carbonate (EMC), and propylene carbonate (PC), which are organic solvents commonly used in electronic devices, have flash points of 143 °C, 17 °C, 102 °C, 23.9 °C, and 132 °C, respectively. All the linear carbonates fall under Class II petroleum or Class III petroleum. It is considered that identifying a safe solvent having high flash point that does not fall under either Class II petroleum or Class III petroleum can reduce management costs.

### EXAMPLE

<Training for Configuring Prediction Device that Predicts Flash Point>

Data pieces for 112 molecular compounds were prepared. These molecular compounds are organic molecular compounds whose molecular structures and flash points are known. The 112 data pieces were collected from publicly known database and literature. In each of the 112 data pieces, the molecular structure of the organic molecular compound is associated with the flash point of the organic molecular compound.

As each explanatory variable set, a combination of 3 explanatory variables from among the 200 RDKit molecular descriptors was used. The number of explanatory variable sets used is 20003=1,313,400. The 1,313,400 explanatory variable sets differ from each other.

The molecular structures of the 112 organic molecular compounds were each converted into values of a plurality of explanatory variables representing the molecular structure of the organic molecular compound, using the RDKit molecular descriptors. Thus, 112 sample data pieces were obtained that were related one-to-one to the molecular compounds. In each sample data piece, the values of the plurality of explanatory variables representing the molecular structure of the organic molecular compound are associated with the value of a response variable representing the flash point of the organic molecular compound.

Of the 112 sample data pieces, 87 data pieces were regarded as the training data. The remaining 25 data pieces were regarded as the test data.

Through training with use of the 87 data pieces, 1,313,400 predictive models associated one-to-one with 1,313,400 explanatory variable sets were constructed. These predictive models are models that predict the flash point with use of linear functions. In each predictive model, the explanatory variable set representing the molecular structure of the organic molecular compound is associated with the response variable representing the flash point of the organic molecular compound.

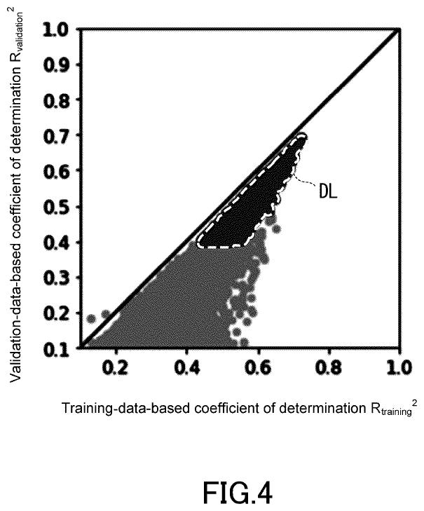

For the 1,313,400 predictive models, the set (R_{training}², R_{validation}²) was calculated in accordance with the description of the sections <Acquisition of validation-data-based evaluation of predictive model> and <Acquisition of training-data-based evaluation of predictive model> above, etc. Specifically, the set (R_{training}², R_{validation}²) was calculated by leave-one-out cross-validation. FIG. 4 is a scatter plot showing the relationship between the training-data-based coefficient of determination R_{training}² of a predictive model according to the example and the validation-data-based coefficient of determination R_{validation}² of the same predictive model. In other words, FIG. 4 is a scatter plot of the sets (R_{training}², R_{validation}²).

In FIG. 4, within the region enclosed by a dotted line DL distributed in the upper part of the diagram, points are plotted that represent the sets (R_{training}², R_{validation}²) for 14,921 predictive models that are neither overfitted nor underfitted and have high correlation from among the 1,313,400 predictive models. In the example, “neither overfitted nor underfitted” means that the absolute value of the difference obtained by subtracting R_{validation}² from R_{training}² is less than 0.106. “High correlation” means that R_{validation}² is greater than 0.4.

In the example, screening was performed to narrow down the 1,313,400 predictive models to the 14,921 predictive models within the dotted line DL. The prediction device according to the example selects, for each organic molecular compound, 10 predictive models from among the 14,921 predictive models in accordance with a selection criterion to construct an ensemble model including the 10 predictive models. The selection criterion in the example is to preferentially select, from among predictive models that satisfy the interpolation condition, 10 predictive models having higher R_{validation}². By including the interpolation condition in the selection criterion, predictive models suited to the molecular structure of the organic molecular compound can be selected, enabling the construction of an ensemble model suited to the molecular structure. Preferentially selecting predictive models having higher R_{validation}² enables the flash point to be predicted with excellent accuracy. The predicted value output by the ensemble model is the arithmetic mean of the predicted values output by the 10 selected predictive models.

### <Evaluation of Prediction Device>

With use of 25 test data pieces, the 14,921 predictive models were evaluated. Based on the test data, the following results were obtained:
a coefficient of determination Rₜₑₛₜ² obtained from the distribution of the actual values and the predicted values was 0.51;
the sum of the absolute errors between the actual values and the predicted values (MAE) was 30.77; and
the standard deviation of the errors between the actual values and the predicted values was 36.48.

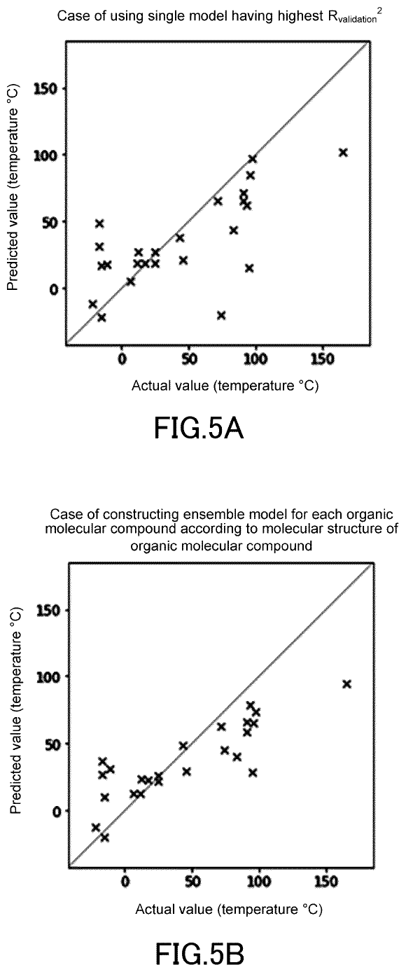

Confirmation was made of the improvement in prediction accuracy that is achieved owing to the selection of, for each organic molecular compound, a plurality of predictive models according to the molecular structure of the organic molecular compound to construct an ensemble model. Specifically, the prediction accuracy of a single model having the highest R_{validation}² from among the 14,921 predictive models was evaluated with use of 25 test data pieces. The results are shown in FIG. 5A. FIG. 5A is a scatter plot showing the relationship between the actual values of the flash point and the predicted values of the flash point obtained in the case of using a single model having the highest R_{validation}². Furthermore, with use of the 25 test data pieces, evaluation was performed on the prediction accuracy achieved in the case of constructing an ensemble model for each organic molecular compound by selecting a plurality of predictive models according to the molecular structure of the organic molecular compound. The results are shown in FIG. 5B. FIG. 5B is a scatter plot showing the relationship between the actual values of the flash point and the predicted values of the flash point obtained in the case of constructing an ensemble model for each organic molecular compound by selecting a plurality of predictive models according to the molecular structure of the organic molecular compound. In FIGS. 5A and 5B, the horizontal axis represents the actual value of the flash point, while the vertical axis represents the predicted value of the flash point.

As understood from FIGS. 5A and 5B, compared to the case of using a single model having the highest R_{validation}², the errors between the actual values and the predicted values are smaller in the case of constructing an ensemble model for each organic molecular compound by selecting a plurality of predictive models according to the molecular structure of the organic molecular compound. Table 1 lists, for both cases, the coefficient of determination Rₜₑₛₜ² obtained from the distribution of the actual values and the predicted values based on the test data, the sum of the absolute errors between the actual values and the predicted values (MAE) based on the test data, and the standard deviation of the errors between the actual values and the predicted values based on the test data. Table 1 demonstrates that, compared to the case of using a single model having the highest R_{validation}², better values of the metrics indicating prediction accuracy are achieved in the case of constructing an ensemble model for each organic molecular compound by selecting a plurality of predictive models according to the molecular structure of the organic molecular compound. It can be understood from Table 1 that, in predicting the flash points of various candidate organic molecular compounds, compared to using a single model having the highest R_{validation}², constructing an ensemble model for each organic molecular compound by selecting a plurality of predictive models according to the molecular structure of the organic molecular compound can more easily ensure prediction reliability across the various candidate organic molecular compounds.

TABLE 1

| | Case where | Case where, for each organic |
|---|---|---|
| | single model | molecular compound, ensemble |
| | having highest | model is constructed according |
| | R_{validation}² | to molecular structure of |
| | is used | organic molecular compound |
| Rₜₑₛₜ² | 0.46 | 0.61 |
| MAE | 26.01 | 23.90 |
| Standard deviation | 35.74 | 30.41 |

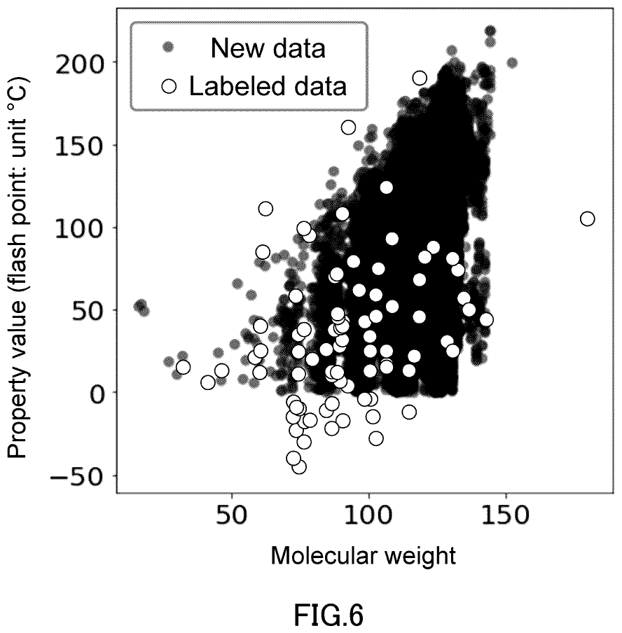

The prediction device configured in the example was used to predict the respective flash points of the 133,885 molecular compounds registered in the QM9 library. The QM9 library contains the molecular structures of small molecular compounds widely used in the field of MI. The prediction results are shown in FIG. 6. FIG. 6 is a scatter plot showing the distribution of the predicted values of the flash points of the molecular compounds contained in the QM9 library, predicted by the prediction device according to the example. In FIG. 6, the horizontal axis represents the molecular weight of the molecular compound, while the vertical axis represents the predicted value of the flash point. “Labeled data” corresponds to the training data. “New data” corresponds to the prediction data related to the molecular compounds contained in the QM9 library, predicted by the prediction device.

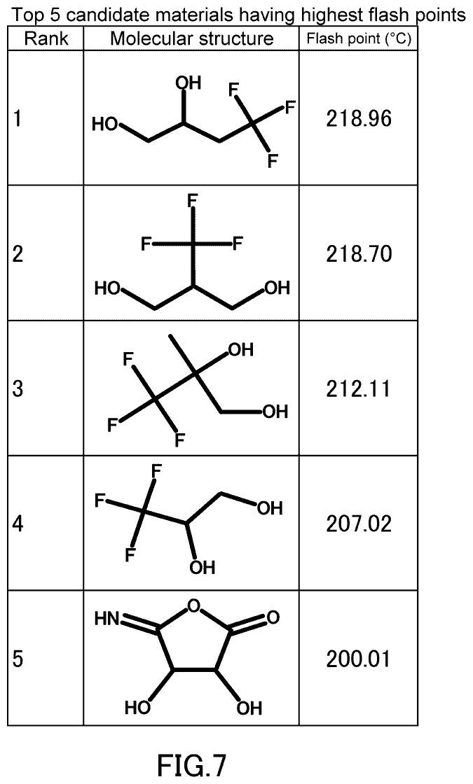

From among the 133,885 molecular compounds, molecular compounds having predicted values of the flash points of 190 °C or more were extracted. The temperature of 190 °C represents the highest flash point from among the above-described 87 data pieces, i.e., the labeled data. As a result of this extraction, 45 molecular compounds were identified from among the 133,885 molecular compounds. The highest predicted value of the flash point from among the 45 molecular compounds was approximately 219 °C. FIG. 7 shows 5 molecular compounds having the higher predicted values of the flash points from among the 45 molecular compounds. The prediction device can be configured to propose these molecular compounds and the predicted values of their flash points. In short, FIG. 7 shows the top 5 molecular compounds having the highest flash points together with the predicted values of their flash points, when molecular compounds that can be proposed in the example are arranged in descending order of predicted flash points.

Furthermore, for the above 14,921 predictive models, a histogram was created based on the number of appearances of each molecular descriptor, and the top 5 molecular descriptors are shown in Table 2 in the form of a table. Specifically, Table 2 lists the top 5 molecular descriptors from the histogram that are included in the predictive models having higher prediction accuracy, specifically having higher R_{validation}². The results shown in Table 2 indicate that a factor related to the total sum of the surface area values of polar regions on the molecular surface (TPSA) significantly contributes to the flash point.

TABLE 2

| Rank | Molecular descriptor | Number of Counts |
|---|---|---|
| 1 | TPSA | 4,591 |
| 2 | VSA_EState3 | 2,425 |
| 3 | fr_Al_OH_noTert | 1,659 |
| 4 | fr_Al_OH | 1,549 |
| 5 | NumHDonors | 1,395 |

Most of the molecular compounds having high flash points shown in FIG. 7 contain fluorine. This is probably because the molecular descriptor, called TPSA, which represents the area of polar regions on the molecular surface, is the major factor contributing to the high flash point (see Table 2).

As understood from the above description, the prediction device according to the example enables the listing, from scarce accumulated past data, of many candidate molecular compounds for which flash points within the target range can be imparted by the many candidate molecular compounds.

The prediction device according to the example is configured to predict the flash point. The prediction device can be configured to predict a different property instead of the flash point. Such a predictive model can be reconstructed by performing training with use of labeled data again. The different property is, for example, viscosity or solubility. The prediction device can also be configured to predict a property of a device containing other elements together with a compound molecule.

Furthermore, as understood from the above description, the prediction device according to the example enables the identification of which factor possessed by the molecular structure of a compound molecule contributes to the property. In the example, as understood from FIG. 6, FIG. 7, and Table 2, the total sum of the surface area values of polar regions on the molecular surface (TPSA) is considered to contribute to the property. In the example, the explanatory variable relating to this factor is the specific explanatory variable.

While the present disclosure has been described through the embodiments and example, the technical scope of the present disclosure is not limited to the scope described in these. It is apparent to those skilled in the art that various modifications or improvements can be added to the embodiments and example. Embodiments with such modifications or improvements can also fall in the technical scope of the present disclosure. For example, instead of generating sample data by the acquisition portion 110, sample data may be provided to the learning device 100 from outside the learning device 100. The prediction and display of the factor predicted to contribute to the property can be omitted. A configuration can also be adopted in which the prediction device 200 transmits an information signal to an external device of the data processing device 10, and based on the information signal, the external device displays a candidate for which the property predicted to be imparted by the candidate is within the target range or displays the factor predicted to contribute to the property.

It should be noted that the operations, procedures, steps, and stages of each process performed by the devices, systems, programs, and methods described in the claims, description, and drawings can be performed in any order unless the order is explicitly specified as “prior to,” “before,” or the like or unless the output of a previous process is used in a subsequent process. Even if operation flows are described using terms such as “first” and “next” in the claims, description, and drawings for convenience, it should not be construed as requiring that the operation flows be performed in this order.

### (Supplementary Description)

The present disclosure provides the following techniques.

### (Technique 1)

A prediction device configured to predict, from a molecular structure of a subject molecular compound, at least one property imparted by the subject molecular compound, the prediction device including:
Z trained predictive models, where Z is a natural number greater than or equal to 2, the Z predictive models being associated one-to-one with Z explanatory variable sets differing from each other, the Z explanatory variable sets each being a combination of p explanatory variables differing from each other from among N explanatory variables, where N is a natural number greater than or equal to 2, the N explanatory variables representing a molecular structure, p is a natural number greater than or equal to 1 and less than N, the Z predictive models each being configured to calculate a value of a response variable representing the property imparted by the subject molecular compound, by substituting a value corresponding to the molecular structure of the subject molecular compound into the explanatory variable set associated with the predictive model;
a model selection portion configured to select, according to the value of the explanatory variable set corresponding to the molecular structure of the subject molecular compound, A predictive models from among the Z predictive models, where A is a natural number greater than or equal to 1 and less than Z; and
a prediction performing portion configured to predict, with use of the A predictive models, the property imparted by the subject molecular compound.

Technique 1 is suitable for performing prediction of a property imparted by a molecular compound according to the molecular structure of the molecular compound.

The at least one property may be one property or may be a plurality of properties. The value of p may be a natural number greater than or equal to 2 and less than N. A may be a natural number greater than or equal to 2 and less than N. A may be a predetermined number. The subject molecular compound may include an organic molecular compound or may consist of an organic molecular compound. The subject molecular compound may include an inorganic molecular compound or may consist of an inorganic molecular compound. The subject molecular compound may be a metal-organic framework (MOF) or may consist of a metal-organic framework. The subject molecular compound may include an organometallic complex or may consist of an organometallic complex. The subject molecular compound may include a macromolecular compound or may consist of a macromolecular compound. The subject molecular compound may include at least two selected from the group consisting of an organic molecular compound, an inorganic molecular compound, a metal-organic framework, an organometallic complex, and a macromolecular compound.

### (Technique 2)

The prediction device according to Technique 1, wherein the Z predictive models are each based on at least one selected from the group consisting of:
a linear function;
a function regularized using ridge regression;
a function regularized using Lasso regression;
a support vector machine;
a neural network;
a maximum likelihood estimation method; and
a Bayesian method.

The predictive models according to Technique 2 are each an example of a predictive model.

### (Technique 3)

The prediction device according to Technique 1 or 2, wherein
when A is a natural number greater than or equal to 2, the prediction performing portion predicts, with use of an ensemble model including the A predictive models, the property imparted by the subject molecular compound.

With use of an ensemble model as in Technique 3, even if there is variation in the performance of individual predictive models, the variation is less likely to affect the property prediction.

### (Technique 4)

The prediction device according to any one of Techniques 1 to 3, further including a providing portion configured to provide the model selection portion with data representing molecular structures of C molecular compounds, where C is a natural number greater than or equal to 1, the molecular structures of the C molecular compounds each being given as the molecular structure of the subject molecular compound, wherein
the providing portion is configured to:
(i) include a molecular generator configured to generate the data representing the molecular structures of the C molecular compounds such that a specified rule defining a permissible molecular structure is satisfied; or
(ii) receive the data representing the molecular structures of the C molecular compounds from a known database.

According to Technique 4, it is possible to predict, for each of C molecular compounds, the property imparted by the molecular compound, where C may be a natural number greater than or equal to 2. The molecular generator in (i) generates data representing the molecular structures of the C molecular compounds, for example, in accordance with a specified algorithm.

### (Technique 5)

The prediction device according to any one of Techniques 1 to 4, wherein
the A predictive models have each been trained with use of a plurality of training data pieces,
the plurality of training data pieces each include a training-purpose input value set that is a combination of values of the N explanatory variables,
the A predictive models each satisfy an interpolation condition that, in a p-dimensional explanatory variable space that represents the explanatory variable set associated with the predictive model, a subject coordinate is interpolated relative to a reference coordinate group,
the subject coordinate is a coordinate defined by the value of the explanatory variable set corresponding to the molecular structure of the subject molecular compound, and
the reference coordinate group is obtained by plotting, for each of the plurality of training data pieces used for training each of the A predictive models, a coordinate defined by the value of the explanatory variable set corresponding to the training data piece.

The A predictive models according to Technique 5 satisfy the interpolation condition, and consequently, have a high likelihood of being suited to the molecular structure of the subject molecular compound.

### (Technique 6)

The prediction device according to Technique 5, wherein
when the Z predictive models include at least one predictive model that satisfies the interpolation condition together with the A predictive models that satisfy the interpolation condition, the A predictive models have higher evaluation than the at least one predictive model.

The A predictive models according to Technique 6 can achieve excellent predictions that merit their high evaluation.

### (Technique 7)

The prediction device according to any one of Techniques 1 to 6, further including a candidate display portion configured to display, when the property predicted by the prediction performing portion to be imparted by the subject molecular compound is within a target range, the subject molecular compound.

According to Technique 7, a user of the prediction device can confirm a molecular compound for which the property within the target range can be imparted by the molecular compound, by viewing the candidate display portion.

### (Technique 8)

The prediction device according to any one of Techniques 1 to 7, further including at least one selected from the group consisting of a factor recording portion and a factor display portion, wherein
the factor recording portion is configured to record factor information, the factor information being based on a specific explanatory variable included in the N explanatory variables and representing a factor predicted to contribute to the property, and
the factor display portion is configured to display the factor information.

According to Technique 8, the factor information can contribute to the creation of a new molecular compound or the creation of a device containing such a molecular compound. The factor information may be the specific explanatory variable itself or may be information that represents the molecular structure represented by the specific explanatory variable in a form other than the specific explanatory variable. The form other than the specific explanatory variable is, for example, a form of letters or a form of chemical structural formulas.

A single display portion may serve as both the candidate display portion and the factor display portion. The single display portion is, for example, the display portion 230 described above. The candidate display portion and the factor display portion may be display portions differing from each other.

### (Technique 9)

A data processing device including:
the prediction device according to any one of Techniques 1 to 8; and
a learning device configured to be provided with a plurality of training data pieces in each of which values of the N explanatory variables representing a molecular structure of a training-purpose molecular compound and a value of a response variable representing the property imparted by the training-purpose molecular compound are associated with each other, wherein
the learning device includes:
   an explanatory variable selection portion configured to select X explanatory variable sets differing from each other, the X explanatory variable sets each being a combination of p explanatory variables differing from each other from among the N explanatory variables, the X explanatory variable sets including the Z explanatory variable sets; and
   a training processing portion configured to construct the Z predictive models associated one-to-one with the Z explanatory variable sets by training with use of the plurality of training data pieces.

According to Technique 9, it is possible to construct Z predictive models.

### (Technique 10)

The data processing device according to Technique 9, wherein
X is _{N}Cₚ.

According to Technique 10, X can easily be made sufficiently large even when N is small. Therefore, the X predictive models are likely to include predictive models suited to the molecular structure of the subject molecular compound.

### (Technique 11)

The data processing device according to Technique 10, wherein
N is a natural number greater than or equal to 4, and
p is 3.

### (Technique 12)

The data processing device according to Technique 10, wherein
N is a natural number greater than or equal to 3, and
p is 2.

In Techniques 11 and 12, p is sufficiently small. This is advantageous from the viewpoint of increasing the calculation speed.

### (Technique 13)

The data processing device according to any one of Techniques 9 to 12, wherein
the learning device further includes a model screening portion configured to select the Z predictive models from among Y predictive models by screening based on at least one metric related to prediction accuracy, where Y is a natural number greater than or equal to Z and less than or equal to X,
the prediction device further includes at least one selected from the group consisting of a factor recording portion and a factor display portion,
the factor recording portion is configured to record factor information, the factor information being based on a specific explanatory variable, the specific explanatory variable being an explanatory variable among the N explanatory variables that has the highest cumulative number of appearances in the Z explanatory variable sets, and
the factor display portion is configured to display the factor information.

In Technique 13, the Z predictive models can be prediction models having high prediction accuracy selected through screening. The specific explanatory variable, which is an explanatory variable having the highest cumulative number of appearances in the Z explanatory variable sets corresponding to the Z predictive models, has a high likelihood of contributing to the property. The factor information based on the specific explanatory variable also has a high likelihood of contributing to the property. The factor information can contribute to the creation of a new molecular compound or the creation of a device containing such a molecular compound. In Technique 13, the screening performed by the model screening portion specifically refers to screening that is performed such that a predictive model having a relatively good value of at least one metric related to prediction accuracy is selected. The “factor” is specifically a factor possessed by the molecular structure. Y may be greater than Z.

In Technique 13, the Z predictive models each may be based on a linear function. According to a linear function, it is easy to understand the correspondence between the explanatory variables related to the molecular structure and the response variable related to the property imparted by the molecular compound. This is advantageous from the viewpoint of increasing the likelihood that the factor information represents the factor that actually contributes to the property. The Z predictive models each may be based solely on a linear function.

### (Technique 14)

A computer-implemented prediction method for predicting, from a molecular structure of a subject molecular compound, at least one property imparted by the subject molecular compound, the prediction method being performed in presence of Z trained predictive models, where Z is a natural number greater than or equal to 2, the Z predictive models being associated one-to-one with Z explanatory variable sets differing from each other, the Z explanatory variable sets each being a combination of p explanatory variables differing from each other from among N explanatory variables, where N is a natural number greater than or equal to 2, the N explanatory variables representing a molecular structure, p is a natural number greater than or equal to 1 and less than N, the Z predictive models each being configured to calculate a value of a response variable representing the property imparted by the subject molecular compound, by substituting a value corresponding to the molecular structure of the subject molecular compound into the explanatory variable set associated with the predictive model, and
the prediction method including:
selecting, according to the value of the explanatory variable set corresponding to the molecular structure of the subject molecular compound, A predictive models from among the Z predictive models, where A is a natural number greater than or equal to 1 and less than Z; and
predicting, with use of the A predictive models, the property imparted by the subject molecular compound.

Technique 14 achieves the same effects as the effects of Technique 1.

### (Technique 15)

A computer-implemented data processing method including:
performing the prediction method according to Technique 14; and
performing a learning method, wherein
the learning method including:
   being provided with a plurality of training data pieces in each of which values of the N explanatory variables representing a molecular structure of a training-purpose molecular compound and a value of a response variable representing the property imparted by the training-purpose molecular compound are associated with each other;
   selecting X explanatory variable sets differing from each other, the X explanatory variable sets each being a combination of p explanatory variables differing from each other from among the N explanatory variables, the X explanatory variable sets including the Z explanatory variable sets; and
   constructing the Z predictive models associated one-to-one with the Z explanatory variable sets by training with use of the plurality of training data pieces.

Technique 15 achieves the same effects as the effects of Technique 9.

### (Technique 16)

A computer program including instructions that, when executed by a computer, cause the computer to perform the prediction method according to Technique 14.

The computer program according to Technique 16 enables the prediction method according to Technique 14 to be performed.

### (Technique 17)

A computer program including instructions that, when executed by a computer, cause the computer to perform the data processing method according to Technique 15.

The computer program according to Technique 17 enables the data processing method according to Technique 15 to be performed.

### (Technique 18)

A non-transitory computer-readable recording medium recording the computer program according to Technique 16.

According to the recording medium according to Technique 18, it is possible to store the computer program according to Technique 16 in a computer that does not have the program recorded and execute the program, thereby performing the prediction method according to Technique 14.

### (Technique 19)

A non-transitory computer-readable recording medium recording the computer program according to Technique 17.

According to the recording medium according to Technique 19, it is possible to store the computer program according to Technique 17 in a computer that does not have the program recorded and execute the program, thereby performing the data processing method according to Technique 15.

The computer can include a processor and a memory. The processor is, for example, a central processing unit (CPU), an application specific integrated circuit (ASIC), or a field programmable gate array (FPGA).

The recording medium is, for example, a semiconductor recording medium, a magnetic recording medium, a magneto-optical recording medium, or an optical recording medium. Examples of semiconductor recording media include hard disk drives (HDDs), secure digital (SD) cards, and universal serial bus (USB) memories. Examples of magnetic recording media include flexible disks. Examples of magneto-optical recording media include magneto optical disks (MOs). Examples of optical recording media include compact discs (CDs), digital versatile discs (DVDs), and solid state drives (SSDs).

### INDUSTRIAL APPLICABILITY

The prediction device, the data processing device, the prediction method, the data processing method, the computer program, and the recording medium of the present disclosure are useful for searching for new materials such as high flash point solvents.

1. A prediction device configured to predict, from a molecular structure of a subject molecular compound, at least one property imparted by the subject molecular compound, the prediction device comprising:
Z trained predictive models, where Z is a natural number greater than or equal to 2, the Z predictive models being associated one-to-one with Z explanatory variable sets differing from each other, the Z explanatory variable sets each being a combination of p explanatory variables differing from each other from among N explanatory variables, where N is a natural number greater than or equal to 2, the N explanatory variables representing a molecular structure, p is a natural number greater than or equal to 1 and less than N, the Z predictive models each being configured to calculate a value of a response variable representing the property imparted by the subject molecular compound, by substituting a value corresponding to the molecular structure of the subject molecular compound into the explanatory variable set associated with the predictive model;
a model selection portion configured to select, according to the value of the explanatory variable set corresponding to the molecular structure of the subject molecular compound, A predictive models from among the Z predictive models, where A is a natural number greater than or equal to 1 and less than Z; and
a prediction performing portion configured to predict, with use of the A predictive models, the property imparted by the subject molecular compound.

2. The prediction device according to claim 1, wherein
the Z predictive models are each based on at least one selected from the group consisting of:
a linear function;
a function regularized using ridge regression;
a function regularized using Lasso regression;
a support vector machine;
a neural network;
a maximum likelihood estimation method; and
a Bayesian method.

3. The prediction device according to claim 1, wherein
when A is a natural number greater than or equal to 2, the prediction performing portion predicts, with use of an ensemble model including the A predictive models, the property imparted by the subject molecular compound.

4. The prediction device according to claim 1, further comprising a providing portion configured to provide the model selection portion with data representing molecular structures of C molecular compounds, where C is a natural number greater than or equal to 1, the molecular structures of the C molecular compounds each being given as the molecular structure of the subject molecular compound, wherein
the providing portion is configured to:
(i) include a molecular generator configured to generate the data representing the molecular structures of the C molecular compounds such that a specified rule defining a permissible molecular structure is satisfied; or
(ii) receive the data representing the molecular structures of the C molecular compounds from a known database.

5. The prediction device according to claim 1, wherein
the A predictive models have each been trained with use of a plurality of training data pieces,
the plurality of training data pieces each include a training-purpose input value set that is a combination of values of the N explanatory variables,
the A predictive models each satisfy an interpolation condition that, in a p-dimensional explanatory variable space that represents the explanatory variable set associated with the predictive model, a subject coordinate is interpolated relative to a reference coordinate group,
the subject coordinate is a coordinate defined by the value of the explanatory variable set corresponding to the molecular structure of the subject molecular compound, and
the reference coordinate group is obtained by plotting, for each of the plurality of training data pieces used for training each of the A predictive models, a coordinate defined by the value of the explanatory variable set corresponding to the training data piece.

6. The prediction device according to claim 5, wherein
when the Z predictive models include at least one predictive model that satisfies the interpolation condition together with the A predictive models that satisfy the interpolation condition, the A predictive models have higher evaluation than the at least one predictive model.

7. The prediction device according to claim 1, further comprising a candidate display portion configured to display, when the property predicted by the prediction performing portion to be imparted by the subject molecular compound is within a target range, the subject molecular compound.

8. The prediction device according to claim 1, further comprising at least one selected from the group consisting of a factor recording portion and a factor display portion, wherein
the factor recording portion is configured to record factor information, the factor information being based on a specific explanatory variable included in the N explanatory variables and representing a factor predicted to contribute to the property, and
the factor display portion is configured to display the factor information.

9. A data processing device comprising:
the prediction device according to claim 1; and
a learning device configured to be provided with a plurality of training data pieces in each of which values of the N explanatory variables representing a molecular structure of a training-purpose molecular compound and a value of a response variable representing the property imparted by the training-purpose molecular compound are associated with each other, wherein
the learning device includes:
an explanatory variable selection portion configured to select X explanatory variable sets differing from each other, the X explanatory variable sets each being a combination of p explanatory variables differing from each other from among the N explanatory variables, the X explanatory variable sets including the Z explanatory variable sets; and
a training processing portion configured to construct the Z predictive models associated one-to-one with the Z explanatory variable sets by training with use of the plurality of training data pieces.

10. The data processing device according to claim 9, wherein
X is _{N}Cₚ.

11. The data processing device according to claim 10, wherein
N is a natural number greater than or equal to 4, and
p is 3.

12. The data processing device according to claim 10, wherein
N is a natural number greater than or equal to 3, and
p is 2.

13. The data processing device according to claim 9, wherein
the learning device further includes a model screening portion configured to select the Z predictive models from among Y predictive models by screening based on at least one metric related to prediction accuracy, where Y is a natural number greater than or equal to Z and less than or equal to X,
the prediction device further includes at least one selected from the group consisting of a factor recording portion and a factor display portion,
the factor recording portion is configured to record factor information, the factor information being based on a specific explanatory variable, the specific explanatory variable being an explanatory variable among the N explanatory variables that has the highest cumulative number of appearances in the Z explanatory variable sets, and
the factor display portion is configured to display the factor information.

14. A computer-implemented prediction method for predicting, from a molecular structure of a subject molecular compound, at least one property imparted by the subject molecular compound, the prediction method being performed in presence of Z trained predictive models, where Z is a natural number greater than or equal to 2, the Z predictive models being associated one-to-one with Z explanatory variable sets differing from each other, the Z explanatory variable sets each being a combination of p explanatory variables differing from each other from among N explanatory variables, where N is a natural number greater than or equal to 2, the N explanatory variables representing a molecular structure, p is a natural number greater than or equal to 1 and less than N, the Z predictive models each being configured to calculate a value of a response variable representing the property imparted by the subject molecular compound, by substituting a value corresponding to the molecular structure of the subject molecular compound into the explanatory variable set associated with the predictive model, and
the prediction method comprising:
selecting, according to the value of the explanatory variable set corresponding to the molecular structure of the subject molecular compound, A predictive models from among the Z predictive models, where A is a natural number greater than or equal to 1 and less than Z; and
predicting, with use of the A predictive models, the property imparted by the subject molecular compound.

15. A computer-implemented data processing method comprising:
performing the prediction method according to claim 14; and
performing a learning method, wherein
the learning method including:
being provided with a plurality of training data pieces in each of which values of the N explanatory variables representing a molecular structure of a training-purpose molecular compound and a value of a response variable representing the property imparted by the training-purpose molecular compound are associated with each other;
selecting X explanatory variable sets differing from each other, the X explanatory variable sets each being a combination of p explanatory variables differing from each other from among the N explanatory variables, the X explanatory variable sets including the Z explanatory variable sets; and
constructing the Z predictive models associated one-to-one with the Z explanatory variable sets by training with use of the plurality of training data pieces.

16. A non-transitory computer-readable recording medium recording a computer program, the computer program comprising instructions that, when executed by a computer, cause the computer to perform the prediction method according to claim 14.

17. A non-transitory computer-readable recording medium recording a computer program, the computer program comprising instructions that, when executed by a computer, cause the computer to perform the data processing method according to claim 15.

18. (canceled)

19. (canceled)
